(19) United States
(12) Patent Application Publication
(10) Pub. No.: US 2010/0159045 A1
(43) Pub. Date: Jun. 24, 2010

(54) COSMETIC USE OF ACTIVE AGENTS FOR STIMULATING THE EXPRESSION OF FN3K AND/OR FN3K RP TO IMPROVE THE SKIN'S BARRIER FUNCTION

(75) Inventors: Christelle Lasserre, Jersey City, NJ (US); Elena Fedorova, Whippany, NJ (US); Yannick Maestro, Martigues (FR)

Correspondence Address: YOUNG & THOMPSON, 209 Madison Street, Suite 500, Alexandria, VA, 22314, US

(73) Assignee: CHANEL PARFUMS BEAUTE, Neuilly Sur Seine (FR)

(21) Appl. No.: 12/664,128

(22) PCT Filed: May 13, 2008
(86) PCT No.: PCT/EP2008/055862

§ 371 Date: Dec. 11, 2009

Related U.S. Application Data
(60) Provisional application No. 60/943,120, filed on Jun. 11, 2007.

(51) Int. Cl. A61K 36/48; A61Q 19/00

(52) U.S. Cl. 424/778

(57) ABSTRACT

A cosmetic process for caring for human skin, intended for preventing or combating the cutaneous signs resulting from non-pathological impairment of the barrier function, includes the topical application to the skin of a composition containing at least one active agent for stimulating the expression of FN3K and/or FN3K RP. The use of this active agent for preventing or combating the cutaneous signs resulting from non-pathological impairment of the barrier function is also disclosed.

The present invention relates to a cosmetic process for caring for human skin, which is intended for preventing or combating the cutaneous signs resulting from a non-pathological impairment of the barrier function, comprising the topical application to the skin of a composition containing an active agent for stimulating the expression of fructosamine-3-kinase (FN3K) or the protein related to fructosamine-3-kinase (FN3K RP).

The skin consists mainly of three layers, namely, starting from the uppermost layer, the epidermis, the dermis and the hypodermis.

The epidermis in particular consists of keratinocytes (predominantly), melanocytes (involved in pigmenting the skin) and Langerhans cells. Its function is to protect the body from the external environment and to ensure its integrity, and especially to halt the penetration of microorganisms or chemical substances, to prevent evaporation of the water contained in the skin and to constitute a barrier against external attack and especially against ultraviolet rays (UV).

To do this, keratinocytes undergo a process of proliferation and then of continuous directed maturation during which the keratinocytes located in the basal layer of the epidermis form, at the final stage of their differentiation, corneocytes, which are totally keratinized dead cells in the form of horny sheaths consisting of proteins and lipids such as ceramides. During this differentiation process, intercorneocytic epidermal lipids are also formed and then organized in the form of bilayers (lamellae) in the stratum corneum, and they participate, with the abovementioned horny sheaths, in the barrier function of the epidermis.

The barrier function of the epidermis may, however, be perturbed under certain climatic conditions (for example under the effect of cold and/or the wind) or under the effect of stress or fatigue, especially, thus promoting the penetration of allergens, irritants or microorganisms, which thus give rise to drying of the skin (the skin loses its permeability, becomes dehydrated and its transepidermal water loss increases), which is liable to give rise to sensations of discomfort such as stinging, tautness, itching, sensations of heating or redness, and also to impair the radiance of the complexion and the suppleness of the skin. Impairment of the skin barrier may also promote the appearance of microchapping or microcracks. Furthermore, a badly formed barrier, resulting from impaired proliferation and differentiation processes, no longer protects the skin against UV radiation or any other type of external attack.

To prevent or correct this phenomenon, it is known practice to apply to the skin cosmetic compositions containing hygroscopic agents, such as sugars or polyols, which are intended to take up the water present in the skin and thus to impede its evaporation. Use has also conventionally been made of fatty substances that allow an occlusive film to be formed on the skin, which contributes towards impeding the evaporation of water. Moreover, these compositions frequently incorporate active agents that act on one or more of the various biological targets involved either in skin regeneration processes, in particular in keratinocyte differentiation, epidermal lipid synthesis and corneocyte cohesion, or in the endogenous synthesis of natural moisturizing factor (NMF) constituents of the skin, in particular in the synthesis of proteoglycans.

Examples of such active agents are especially ∝- and β-hydroxy acids, especially lactic acid, glycolic acid and salicylic acid; urea; and aminosulfonic compounds.

However, there always remains the need to propose novel cosmetic active agents for reinforcing the skin's barrier function to prevent and/or reduce the sensations of cutaneous discomfort, stinging, tautness, itching, sensations of heating or redness and/or the appearance of microchapping or microcracking and/or the loss of radiance of the complexion or dull complexion and/or the loss of suppleness of the skin and/or to improve the protection of the epidermis against UV.

In addition, given the ever-increasing search by consumers for natural products containing the fewest possible synthetic ingredients, and the increasingly burdensome regulatory constraints on compounds derived from the chemical industry, it would be desirable for these cosmetic active agents to be of plant origin.

Now, the Applicant has, to its credit, shown, unexpectedly, that it is possible to act on two novel biological targets, namely fructosamine-3-kinase and fructosamine-3-kinase-related protein, to combat impairment of the barrier function. The Applicant has also shown that FN3K in skin diminishes with increasing age and that the absence of FN3K in reconstructed skins had the same consequence as the effect of glycation on catalase expression and on epidermal thickness. The Applicant has also, to its credit, developed an appropriate screening test for selecting active agents acting on this target and for identifying plant extracts that respond to this test, thus making it possible to satisfy the abovementioned needs.

Fructosamine-3-kinase (referred to hereinbelow as FN3K) is an enzyme expressed in the liver, which has been isolated from human erythrocytes and which is capable of phosphorylating fructosamines, produced by the non-enzymatic reaction of proteins with glucose (Maillard reaction or glycation), to form an unstable product leading to the regeneration of the non-glycated amine (Delpierre G et al., Biochem. Soc. Trans. 203 Dec; 31 (Pt 6): 1354-7). FN3K RP is also involved in the deglycation process. These enzymes are thus considered as potential targets for combating the glycation of proteins and the formation of advanced glycation products (AGEs) that contribute especially towards complications diabetes, of osteoarthritis (degeneration of cartilage) and to amyloidosis. To the Applicant's knowledge, however, it has never been suggested that this enzyme is expressed in the epidermis or, all the less so, that it might intervene in the maturation process (proliferation and then differentiation) of keratinocytes.

Thus, cosmetic active agents that stimulate the expression of fructosamine-3-kinase (FN3K) or its related protein (FN3K RP) have never yet been disclosed, and even less so has it been suggested to use them in topical application on human skin.

One subject of the present invention is thus a cosmetic process for caring for human skin, which is intended for preventing or combating the cutaneous signs resulting from non-pathological impairment of the barrier function, comprising the topical application to the skin of a composition containing at least one active agent for stimulating the expression of fructosamine-3-kinase and/or its related protein (FN3K RP).

A subject of the present invention is also the cosmetic use of an active agent for stimulating the expression of fructosamine-3-kinase and/or FN3K RP, for preventing or combating the cutaneous signs resulting from non-pathological impairment of the barrier function.

As a preamble, it is pointed out that the expression “active agent for stimulating the expression of FN3K and/or FN3K RP” means a compound or (especially in the case of a botanical extract) a mixture of compounds capable of stimulating the expression of FN3K and/or FN3K RP relative to an untreated control, which is determined in particular by means of the real-time polymerase chain amplification method (RT-PCR) in cells or in reconstructed skins as described in the examples below.

The active agent for stimulating the expression of FN3K and/or FN3K RP may be used in a proportion of from 0.00001% to 10% by weight, preferably in a proportion of from 0.0001% to 5% by weight and more preferably in a proportion of from 0.001% to 1% by weight relative to the total weight of the composition.

The active agents that may be used according to the invention are advantageously botanical extracts, i.e. active agents obtained by extraction, using any type of solvent, of any part of a plant such as bark, wood, roots, rhizomes, stalks, leaves, fruit or flowers, for example.

An example of such active agents especially comprises an alcoholic extract of Butea frondosa blossom. This extract may be obtained by alcoholic extraction using at least one monoalcohol such as ethanol, methanol or isopropanol and/or at least one glycol such as propylene glycol or dipropylene glycol, optionally mixed with water. The extraction is then performed in the absence of any other solvent. In general, in the case of aqueous-alcoholic solvents, it is preferable for the volume ratio of the alcohol to water to be between 70% and 96%.

In general, the extraction may be performed on fresh or dried flowers, optionally chopped or ground, in the usual manner. The extraction is generally performed by immersing or gently shaking the flowers in one or more of the solvents mentioned above at temperatures ranging, for example, from room temperature to 100° C. and advantageously from 30 to 70° C., for a time of about 30 minutes to 12 hours and preferably from 1 to 8 hours. The solution is then preferably filtered so as to remove the insoluble substances of the plant. The solvent is also, where appropriate, removed if it is a volatile solvent, for instance ethanol, methanol or isopropanol. This extraction step is common in the field of plant extracts and a person skilled in the art is capable of adjusting the reaction parameters thereof on the basis of his general knowledge.

After this extraction step, an extract of Butea frondosa flowers is obtained, which may then, according to an advantageous aspect of the invention, be subjected to a decolorizing step, especially using active charcoal in the presence of a solvent. The weight of active charcoal is preferably between 0.5% and 50% of the weight of the extract. One or more solvents chosen from water, C₁-C₄ alcohols such as methanol, ethanol or isopropanol, polar organic solvents such as propylene glycol or dipropylene glycol, or any other solvent that is common in the field, may especially be used. The volatile solvents may then be removed under reduced pressure.

The active agent for stimulating the expression of FN3K and/or FN3K RP is used according to the invention for cosmetic purposes, to prevent or combat the cutaneous signs resulting from non-pathological impairment of the barrier function. The process according to the invention may thus especially be used to preserve and/or reinforce the skin's barrier, especially to combat the cutaneous signs resulting from perturbed but non-pathological barrier function, including roughness of the skin, discomfort including redness, tautness, stinging and itching, the appearance of microchapping or microcracking, the loss of radiance of the complexion or dull complexion, the loss of suppleness of the skin, and also to improve the protection of the epidermis against UV. It may advantageously be used to moisturize the skin and/or protect it against drying. The moisturizing effect of the composition used according to the invention may especially be measured by corneometry, according to usual techniques that are well known to those skilled in the art.

The active agent used according to the invention, or the composition used in the process according to the invention, are preferably applied to non-pathological dry skin. They may advantageously be applied to the skin of the face, the neck and possibly the neckline or, as a variant, to any part of the body.

The composition containing this active agent may be applied in the morning and/or in the evening, to the entire face, the neck and optionally the neckline or even the body.

The composition used according to the invention generally comprises, besides the active agent described previously, a physiologically acceptable and preferably cosmetically acceptable medium, i.e. a medium that is suitable for use in contact with human skin without any risk of toxicity, incompatibility, instability or allergic response and especially that does not cause any sensations of discomfort (redness, tautness, stinging, etc.) that are unacceptable to the user.

This medium generally contains water and optionally other solvents such as ethanol.

The composition used according to the invention may be in any form that is suitable for topical application to the skin and in particular in the form of an oil-in-water, water-in-oil or multiple emulsion (W/O/W or O/W/O), which may optionally be microemulsions or nanoemulsions, or in the form of an aqueous dispersion, a solution, an aqueous gel or a powder. It is preferable for this composition to be in the form of an oil-in-water emulsion.

This composition is preferably used as a care and/or cleansing product for facial and/or bodily skin and it may especially be in the form of a fluid, a gel or a mousse, conditioned, for example, in a pump-dispenser bottle, an aerosol or a tube, or in the form of cream conditioned, for example, in a jar. As a variant, it may be in the form of a makeup product and in particular a foundation or a loose or compact powder.

It may contain various adjuvants, such as at least one compound chosen from:
oils, which may be chosen especially from: linear or cyclic, volatile or non-volatile silicone oils, such as polydimethylsiloxanes (dimethicones), polyalkylcyclosiloxanes (cyclomethicones) and polyalkylphenylsiloxanes (phenyl dimethicones); synthetic oils such as fluoro oils, alkylbenzoates and branched hydrocarbons such as polyisobutylene; plant oils and especially soybean oil or jojoba oil; and mineral oils such as liquid petroleum jelly;
waxes such as ozokerite, polyethylene wax, beeswax or carnauba wax;
silicone elastomers obtained especially by reaction, in the presence of a catalyst, of a polysiloxane containing at least one reactive group (especially hydrogen or vinyl) and bearing at least one alkyl group (especially methyl) or phenyl, in a terminal and/or side position, with an organosilicone such as an organohydrogenopolysiloxane;
surfactants, preferably emulsifying surfactants, whether they are nonionic, anionic, cationic or amphoteric, and in particular fatty acid esters of polyols such as fatty acid esters of glycerol, fatty acid esters of sorbitan, fatty acid esters of polyethylene glycol and fatty acid esters of sucrose; fatty alkyl ethers of polyethylene glycol; alkylpolyglucosides; polysiloxane-modified polyethers; betaine and derivatives thereof; polyquaterniums; ethoxylated fatty alkyl sulfate salts; sulfosuccinates; sarcosinates; alkyl and dialkyl phosphates, and salts thereof; and fatty acid soaps;
co-surfactants such as linear fatty alcohols and in particular cetyl alcohol and stearyl alcohol;
thickeners and/or gelling agents, and in particular crosslinked or non-crosslinked, hydrophilic or amphiphilic homopolymers and copolymers, of acryloylmethylpropanesulfonic acid (AMPS) and/or of acrylamide and/or of acrylic acid and/or of acrylic acid salts or esters; xanthan gum or guar gum; cellulose derivatives; and silicone gums (dimethiconol);
organic screening agents, such as dibenzoylmethane derivatives (including butylmethoxydibenzoyl-methane), cinnamic acid derivatives (including ethylhexyl methoxycinnamate), salicylates, para-aminobenzoic acids, β,β′-diphenyl acrylates, benzophenones, benzylidenecamphor derivatives, phenylbenzimidazoles, triazines, phenyl-benzotriazoles and anthranilic derivatives;
inorganic screening agents, based on mineral oxides in the form of coated or uncoated pigments or nanopigments, and in particular based on titanium dioxide or zinc oxide;
dyes;
preserving agents;
fillers, and in particular powders with a soft-focus effect, which may be chosen especially from polyamides, silica, talc, mica and fibers (especially polyamide fiber or cellulose fiber);
sequestrants such as EDTA salts;
fragrances;
and mixtures thereof, without this list being limiting.

Examples of such adjuvants are especially mentioned in the CTFA dictionary (International Cosmetic Ingredient Dictionary and Handbook published by The Cosmetic, Toiletry and Fragrance Association, 11th edition, 2006), which describes a wide variety, without limitation, of cosmetic and pharmaceutical ingredients usually used in the skincare industry, that are suitable for use as additional ingredients in the compositions according to the present invention.

The composition used according to the invention may also provide additional benefits, including calmative or anti-inflammatory activity, bleaching or depigmenting activity, anti-aging activity and/or cleansing activity.

The composition used according to the invention may also comprise active agents other than those that stimulate the expression of FN3K and/or FN3K RP, and in particular at least one active agent chosen from: keratolytic agents and in particular ∝-hydroxy acids such as glycolic acid, lactic acid and citric acid, and esters or salts thereof; β-hydroxy acids such as salicylic acid and derivatives thereof; agents for increasing keratinocyte differentiation and/or cornification, either directly or indirectly by stimulating, for example, the production of β-endorphins, such as extracts of Thermus thermophilus or extracts of bean husks of Theobroma cacao, water-soluble extracts of corn, peptide extracts of Voandzeia substerranea and niacinamide; epidermal lipids and agents for increasing the synthesis of epidermal lipids, either directly or by stimulating certain β-glucosidases that modulate the deglycosylation of lipid precursors such as glucosyl ceramide to ceramides, such as phospholipids, ceramides, lupin protein hydrolyzates and dihydrojasmonic acid derivatives; humectants, such as polyols and in particular glycerol, glycosaminoglycans such as hyaluronic acid, sugars and alkyl esters thereof, amino acids such as glycine, arginine, histidine, alanine, threonine, lysine, glutamic acid, taurine, proline, serine and derivatives thereof, pyrrolidonecarboxylic acid (PCA) and salts thereof, urea and derivatives thereof, ectoin, glucosamine, creatine, choline, betaine, mineral salts such as chlorine, sodium, potassium, calcium, magnesium, zinc, manganese or phosphate salts and humectant synthetic polymers such as methacryloyloxyethylphosphorylcholine homopolymers and copolymers, and glyceryl(meth)acrylate homopolymers and copolymers; or at least one active principle that stimulates the expression of matriptase MT/SP1 such as an extract of Cananga odorata Hook, or Cedrelopsis grevei, or Cistus ladaniferus L. or caroba pulp (Ceratonia siliqua); antioxidants and/or free-radical scavengers and/or anti-pollution agents, such as tocopherol and esters thereof, ascorbic acid and the alkyl and phosphoryl esters thereof and certain extracts of plants or algae and in particular of Thermus thermophilus; and mixtures thereof, without this list being limiting.

The combination of active agents for stimulating the expression of FN3K and/or FN3K RP with one or more of the agents described above makes it possible advantageously to combine in the same formula the effects of these two types of active agent and thus to obtain maximum and long-lasting moisturization of the skin.

The invention will now be illustrated by the non-limiting examples that follow.

### EXAMPLES

### Example 1

### Preparation of an extract of Butea frondosa

1) Aqueous-Alcoholic Extraction

1.110 kg of Butea frondosa flowers are ground using a knife mill (Retsch) and loaded into a 20 l glass reactor equipped with a reflux condenser.

7.8 l of 96% (volume/volume) ethanol are added.

Heating of the reactor is started at 50° C. Heating is continued for 5 hours.

The material is then filtered so as to remove the ground material of Butea frondosa flowers. The filtrate is recovered.

The solvent is then evaporated off on a rotary evaporator under vacuum.

0.183 kg of extract of Butea frondosa flowers is thus recovered.

The yield for this operation is 16.5%.

2) Decolorization of the Extract

The oleoresin is hot-washed with 96% (volume/volume) ethanol and active charcoal:

183 g of oleoresin are mixed with 1500 ml of 96% ethanol and 24 g of active charcoal. The mixture is stirred vigorously for 2 hours at 50-60° C. and is then left to stand at room temperature for 2 hours. After filtering the solution through a Büchner funnel, the primary filtrate is recovered.

This filtrate is then filtered again on a conical filter in order to remove the final residues of active charcoal, and the ethanol is then evaporated off using a rotary evaporator under vacuum.

The yield for this decolorization operation is 68%.

The total yield for the process is 11.2%.

Before being tested on skin cells as described in Examples 2 to 5 below, the extract is diluted to 20% in propylene glycol.

### Example 2

### Test of Stimulation of the Expression of the Messenger RNA (mRNA) of FN3K and FN3K RP in Normal Keratinocytes with an Extract of Butea frondosa

Protocol:

The effect of the botanical extract of Example 1 on the expression of the mRNA of FN3K and/or FN3K RP was evaluated on keratinocytes.

Keratinocytes derived from neonatal foreskins (Clonetics, Calif., USA) were inoculated in 6-well plates and cultured in keratinocyte growth culture medium (KBM, Clonetics), i.e. a modified culture medium supplemented with human recombinant EGF, insulin, hydrocortisone, bovine pituitary extract, gentamycin and amphotericin B. After culturing for 24 hours in an oven at 37° C., the confluent cells were washed with PBS buffer (Invitrogen, CA) and incubated with specific basic medium (KBM, Clonetics) containing the extract to be tested, for 24 hours, at increasing concentrations. After studying the cytotoxicity of the extract, its activity was evaluated.

To quantify the expression of the messenger RNA of FN3K and of FN3K RP in a treated sample relative to an untreated sample, real-time polymerase chain amplification (RT-PCR) was used. The results were normalized relative to the expression of domestic genes of these samples and corrected as regards the differences in efficacy of PCR. The results were expressed in terms of the number of times of increase or of decrease of expression of the target gene FN3K or FN3K RP in the treated sample.

The cDNA/mRNA sequences of the genes investigated were obtained from Genbank.

Domestic Gene: PBGD

All the PCR primers were obtained using the scientific publication of Conner, J., et al., 2005. Ann. N.Y. Acad. Sci. 1043: 824:836. The keratinocytes were treated with various concentrations of extracts in triplicate for 24 hours. The mRNA was isolated using the reagent Qiagen RNeasy kit and quantified using the Quantlt kit (Invitrogen, CA).

Reverse transcription was performed using the gene Amp RNA PCR kit (Applied Biosystems) according to the manufacturer's recommendations.

The real-time PCR measurement was performed using the iCYCLER IQ machine (Bio-rad, CA) with SYBR Green I detection.

In all the tests, the cDNA was amplified using a standardized program. Each sample was charged with supermix IQ SYBR Green I, water and primer (stock). The final amount of cDNA per reaction corresponded to 75 ng of total RNA used for the reverse transcription.

The relative quantification of the expression of the target gene was performed using the Pfaffl mathematical model (Pfaffl, MW, Nucleic Acids Res. 29(9), p. E45, 2001).

The positive results were confirmed using cells from two different donors.

Results:

The results are given in Tables 1 and 2 below:

TABLE 1

| | | Stimulation of | Standard |
|---|---|---|---|
| | Concentration⁽¹⁾ | FN3K mRNA | deviation |
| Untreated | — | 1.05 | 0.01 |
| keratinocytes | | | |
| Butea frondosa | 0.02% | 1.26 | 0.07 |
| | 0.1% | 7.36 | 0.75 |

TABLE 2

| | Stimulation of | Standard |
|---|---|---|
| Active agent tested | FN3K RP mRNA | deviation |
| Untreated | 1.04 | 0.01 |
| keratinocytes | | |
| Butea frondosa at 0.1%⁽¹⁾ | 2.23 | 0.085 |

⁽¹⁾the concentrations of the extracts are expressed as the weight of crude extract per weight of preparation

It emerges from this test that the Butea frondosa extracts make it possible to stimulate the expression of the mRNA of FN3K and of FN3K RP in normal keratinocytes.

### Example 3

### Test of Stimulation of the Expression of the Protein FN3K in a Skin Equivalent with an Extract of Butea frondosa

Protocol:

The effect of the botanical extract of Example 1 on the expression of fructosamine-3-kinase (FN3K) was evaluated in a model of reconstructed skin.

This model was prepared in the following manner: a collagen solution containing type I collagen from rat tail (BD, CA), 10×DMEM medium (Invitrogen, CA), sodium bicarbonate (Invitrogen) and fibroblasts was poured into 24 mm cell culture inserts (Falcon, Becton Dickinson, Schwechat, Austria), which were placed in six-well plates (Falcon). After two hours at 37° C., the gels were equilibrated in KGM (Clonetics) at 37° C. in an environment containing 5% CO₂/95% air, in a humidified incubator. After two hours, KGM containing keratinocytes was added to the gel. After immersing the culture overnight, the medium was replaced with serum-free keratinocyte medium (SKDM, which is a medium rich in Ca²⁺consisting of KGM without bovine pituitary extract, transferrin from Sigma, BSA from Sigma and L-ascorbic acid from Sigma) outside the insert, and the keratinocytes were maintained at the air-liquid interface. The culture medium of the reconstructed skins was replaced every two days with preheated fresh SKDM, and culturing was continued for up to seven days, with or without the active agent at various concentrations.

The reconstructed skins were then prepared in order to be analyzed by immunofluorescence. Slices 7 μm thick were cut from the reconstructed skins, fixed with paraformaldehyde and then frozen. The nonspecific bonding of the slices was blocked with serum (bovine serum albumin). The samples of reconstructed skin thus prepared were incubated with an anti-FN3K antibody (Santa Cruz, Calif.), and then labeled in a second step with a second antibody complexed with a fluorescent agent (Alexa Fluor 546 anti-rabbit antibody, Molecular Probes, UK). Detection was performed by immunofluorescence. The slides were examined using a Leica microscope.

Results:

It was observed that the extract of Butea frondosa at 0.02% reproducibly stimulated the expression of FN3K visibly on the viable epidermis of the skin equivalent. These results were confirmed using reconstructed skins obtained from two donors.

### Example 4

### Evaluation of the expression of FN3K with age

Protocol:

The variation in expression of the FN3K protein was evaluated by immunohistochemistry (IHC), using freezed skin samples from 3 to 5 donors of various ages. Staining was performed on cryosections of 5 μl from 2 age groups (30-40 years old and 60-70 years old), with anti-FN3K antibodies (Santa Cruz, Calif.) and secondary antibodies (Jackson Immunoresearch Labs, PA).

The extent of staining was assessed on 6 sections from each donor, and a visual assessment of the sections was made using a scale from 0.5 to 5 in absolute value.

Results:

Evaluation of FN3K staining in young skins was 4.67 (±0.33) and that in elder skins was 1.83 (±0.66). This demonstrates that the amount of FN3K diminishes with increasing age.

### Example 5

### Effect of FN3K and FN3K RP Silencing on Keratinocyte Proliferation

Keratinocytes derived from neonatal foreskins of a single donor (Clonetics, Calif.) were cultured at 37° C. in an environment containing 5% CO₂/95% air, in a humidified incubator and in a growth medium suitable for growing keratinocytes (KFM, Clonetics). These keratinocytes were then transfected with a silencer RNA specific for FN3K and FN3K RP using the transfectant NeoFX and by performing the siPORT Neo FX transfection protocol described by the supplier of the silencer RNA (Ambion, Tex.). Three different RNAs that inactivate FN3K or FN3K RP were tested. The transfected or non-transfected cells (negative control) were recultured for 5 days and then analyzed by RT-PCR using the same method as that described in Example 2.

The results obtained made it possible to demonstrate that inactivation of expression of FN3K and/or of FN3K RP through their respective silencer RNAs induced a strong reduction of the proliferation of normal human keratinocytes when FN3K is silenced and a less important reduction when FN3K RP is silenced.

### Example 6

### Study of Epidermal Thickness of Reconstructed Skins without FN3K (Silenced FN3K)

Protocol:

Reconstructed epidermal skins were produced from human keratinocytes that were normal or FN3K-silenced using the siRNA technique or transfected with scramble siRNA as an experimental control. After 6 days of culture, the reconstructed skins were stained with H&E (hematoxyline and eosine) to assess the morphology of the reconstructed skins. Epidermal thickness was then measured. For each point, 150 measurements were performed of three different skins prepared from keratinocytes of two different donors.

Results:

Silencing of the FN3K siRNA results in a reduction in epidermal thickness revealing a reduction in reconstructed skin growth and viability. FN3K is thus an essential element in the formation of an epidermis.

TABLE 3
Epidermis thickness in μm

| | | | Experimental |
|---|---|---|---|
| Control/ | SiARN1/ | siARN2/ | control/ |
| Standard | Standard | Standard | Standard |
| deviation | deviation | deviation | deviation |
| 408.8 +/− 137.46 | 300.8 +/− 77.34 | 348.3 +/− 97 | 489.1 +/− 157.7 |

### Example 7

### Expression of Catalase in FN3K-Silenced Reconstructed Skins, Compared with Glycation-Induced Reconstructed Skins

Protocol:

Glycation was induced in cultured reconstructed skins by adding 250 μg of methylglyoxal. The effects of glycation and FN3K-silencing on catalase expression in reconstructed skins were assessed by immunohistochemistry.

Results:

Catalase expression significantly decreases in glycated reconstructed skins and in FN3K-silenced skins. This demonstrates the importance of FN3K in the normal functioning of the epidermis and its protective effect against radicals.

### Example 8

### Stimulation of FN3K Messenger RNA in Normal Keratinocytes Treated with UVB Rays

Keratinocytes derived from neonatal foreskins (Clonetics, Calif.) were inoculated in 6-well plates and cultured in culture medium for keratinocyte growth (KBM, Clonetics), namely a modified culture medium supplemented with human recombinant EGF, insulin, hydrocortisone, bovine pituitary extract, gentamycin and amphotericin B. After culturing for 24 hours in an oven at 37° C., the confluent cells were washed with PBS buffer (Gibco) and then irradiated with UVB using a BioSun machine (Vilber Lourmat) with different doses of UVB and finally incubated for 24 hours in standard keratinocyte culture medium (Cambex, Md.).

To quantify the expression of the FN3K mRNA, a protocol identical to that described in Example 2 was used.

The positive results were confirmed using cells from two different donors.

Results:

The results are given in Table 4 below:

TABLE 4

| | Dose of | Stimulation of | Standard |
|---|---|---|---|
| | UVB | FN3K mRNA | deviation |
| Untreated | — | 1.01 | 0.04 |
| keratinocytes | | | |
| UVB-treated | 5 mJ/cm² | 1.38 | 0.27 |
| keratinocytes | 10 mJ/cm² | 1.71 | 0.19 |
| | 20 mJ/cm² | 3.00 | 1.01 |
| | 30 mJ/cm² | 4.63 | 1.54 |

It emerges from this test that the UVB irradiation makes it possible to stimulate the expression of FN3K mRNA in normal keratinocytes and that this stimulation is proportional to the dose of UVB received by the keratinocytes. Increasing the synthesis of FN35 by the keratinocytes is thus a first means of defense established by the skin to protect itself against UV rays.

### Example 9

### Cosmetic Composition

The following composition may be prepared in a manner that is conventional for those skilled in the art. The amounts indicated below are expressed as weight percentages. The ingredients in upper case are identified in accordance with the INCI name.

| | |
|---|---|
| Tetrasodium EDTA | 0.05% |
| POLYGLYCERYL METHACRYLATE & | 5.00% |
| PROPYLENE GLYCOL⁽¹⁾ | |
| Glycerol | 6.00% |
| Aqueous-phase gelling agents | 5.50% |
| Nonionic emulsifiers | 4.00% |
| Cetearyl alcohol | 2.00% |
| Emollients | 17.00% |
| Tocopheryl acetate | 0.50% |
| Preserving agents | 2.20% |
| Extract of Butea frondosa⁽²⁾ | 0.05% |
| Sodium hyaluronate | 5.00% |
| Fragrance | qs |
| Dyes | qs |
| Water | qsp 100.00% |

⁽¹⁾LUBRAJEL MS ® from Guardian Laboratories
⁽²⁾as described in Example 1 and then diluted to 80% by weight in dipropylene glycol

This composition, in the form of an oil-in-water emulsion, may be applied daily, morning and/or evening, to facial skin to moisturize it and make it supple, smooth and luminous.

1-10. (canceled)

11. Cosmetic process for preventing or combating the cutaneous signs resulting from non-pathological impairment of the barrier function, comprising the topical application to the skin of a composition containing at least one active agent that stimulates the expression of fructosamine-3-kinaase (FN3K) and/or its related protein (FN3K RP).

12. Process according to claim 11, wherein the active agent that stimulates the expression of FN3K and/or FN3K RP is a botanical extract.

13. Process according to claim 12, wherein the active agent is an alcoholic extract of Butea frondosa blossom.

14. Process according to claim 13, wherein the extract is obtained by extraction using at least one monoalcohol and/or at least one glycol, optionally mixed with water.

15. Process according to claim 11, which is intended for preserving and/or reinforcing the skin barrier.

16. Process according to claim 11, which is intended for moisturizing the skin and/or for protecting it against drying out.

17. Process according to claim 11, wherein the said signs are chosen from: skin roughness, redness, tautness, stinging, itching, the appearance of microchapping or microcracking, the loss of radiance of the complexion and/or the loss of suppleness of the skin.

18. Process according to claim 11, which is intended for improving the protection of the epidermis against UV rays.

19. Process according to claim 11, wherein said skin is non-pathological dry skin.
